(19) United States
(12) Patent Application Publication
(10) Pub. No.: US 2007/0184481 A1
(43) Pub. Date: Aug. 9, 2007

(54) Assay For Transcriptionally Active Methylated Promoters

(75) Inventors: George Blanck, Tampa, FL (US); Melissa I. Niesen, Tampa, FL (US); Aaron R. Osborne, Lutz, FL (US)

Correspondence Address: SMITH HOPEN, PA, 180 PINE AVENUE NORTH, OLDSMAR, FL, 34677, US

(73) Assignee: UNIVERSITY OF SOUTH FLORIDA, Tampa, FL (US)

(21) Appl. No.: 11/735,706

(22) Filed: Apr. 16, 2007

Related U.S. Application Data
(63) Continuation of application No. PCT/US05/37673, filed on Oct. 18, 2005.

(51) Int. Cl. C12Q 1/68; C12Q 1/66

(52) U.S. Cl. 435/6; 435/8

(57) ABSTRACT

A method of identifying genes that can be activated when methylated; as well as proteins that mediate such activation. The method allows identification of HLA-DRA silencing agents expected to be of therapeutic value in the treatment of inflammation associated with HLA-DRA expression.

### CROSS REFERENCE TO RELATED APPLICATIONS

This application is a continuation of International Application No.

PCT/US2005/037673 filed Oct. 18, 2005, which claims priority to Provisional U.S. patent application Ser. No. 60/619,265 filed Oct. 15, 2004, which is fully incorporated herein by reference.

### GOVERNMENT SUPPORT

Research relating to the present application was supported by NIH grant R01 CA81497. Accordingly, the federal government may have rights in the present invention.

### BACKGROUND OF THE INVENTION

The present invention relates to the field of gene regulation. In particular, the invention provides novel screening systems for identifying test agents that modulate expression of methylation-dependent repressed genes.

Methylation of DNA cytosine residues is strongly associated with higher order chromatin formation, heterochromatin, and repression of RNA transcription. Several molecular mechanisms leading to transcriptional repression, as a result of DNA methylation, have been elucidated. For example, MeCP2, one of a family of proteins with a methyl-DNA binding domain (MBDs), binds to 5-methylcytosines recruiting a histone deacetylase, which in turn leads to the dissociation of transcriptional activation proteins from the histone constituted-nucleosome, nucleosome stabilization, recruitment of proteins that mediate chromatin condensation, and the repression of transcription.

The roles of eukaryotic DNA methylation in the repression of mRNA transcription and in the formation of higher order, heterochromatic chromatin structure, which prevents mRNA transcription over long, multi-gene chromosomal distances, have been extensively elucidated over the past several years. However, the role of DNA methylation in transcriptional activation remains a mystery. In particular, it is not clear whether DNA methylation that leads to the repression of transcription can also be compatible with transcriptional activation. And, it is not known whether the transcriptional activation of methylated DNA is promoter-specific, involves sequence specific DNA binding proteins, or is facilitated by the methylation.

In several rare cases, DNA methylation has been shown to enhance transcription. DNA methylation of the far upstream region of the IL-8 gene and methylation of the egr-2 intron are associated with increased promoter activity. The mechanism of these effects is unknown, and no DNA binding proteins involved in these processes have been identified. Other studies have indicated that methylated DNA can be transcribed, apparently in the absence of proteins that directly mediate transcriptional repression. However, none of these studies involve a transition from repressed to activated methylated DNA. Recently, Lembo et al. have identified a protein that facilitates a transition from MBD-mediated repression to MBD-mediated activation. However, the molecular mechanism that specifies a transition from repressed methylated DNA to methylated DNA that becomes or remains transcriptionally active, for a given promoter, remains unknown.

What is needed is a way to assay for the activation of a methylated gene promoter. For example, assaying for transcription of the methylated HLA-DRA gene may provide a way to obtain novel anti-inflammatory drugs because the gene is sometimes methylated and transcribed.

### SUMMARY OF INVENTION

In one embodiment, the invention includes an assay for drugs that interfere with HLA-DRA gene expression, thus reducing inflammation.

In another embodiment, the invention includes an assay for transcriptional activation of methylated DNA for any gene. Accordingly, the present invention provides a method of identifying genes that can be activated when methylated; as well as proteins that mediate such activation. In one embodiment the assay is used as a positive control for future experiments, ie, to ensure validation and quality control for future tests, experiments, assays, etc.

In another embodiment, the inventors show that the sequence specific DNA binding protein, RFX, previously shown to mediate the transition from an inactive to an active chromatin structure, preferentially activates a methylated promoter. RFX is capable of mediating enhanceosome formation on a methylated promoter, thereby mediating a transition from a methylation-dependent repression of the promoter to a methylation-dependent activation of the promoter. These results show that methylated DNA is specifically activated by sequence specific DNA binding proteins and provide novel roles for DNA methylation and sequence specific DNA binding proteins in transcriptional activation.

### BRIEF DESCRIPTION OF THE DRAWINGS

For a fuller understanding of the nature and objects of the invention, reference should be made to the following detailed description, taken in connection with the accompanying drawings, in which:

FIG. 1 is a graph of experiment results showing DNA methylation represses transfected HLA-DRA promoter.

FIGS. 2A through 2D are graphs of experiment results showing RFX activation of methylated pDRA.

FIGS. 3A and 3B are graphs of experiment results showing RFX facilitates CIITA activation of pDRA.

FIGS. 4A through 4C are graphs of experiment results showing RFX does not activate unmethylated pDRA and does not decrease the level of pDRA methylation.

FIG. 4D is a pDRA map indicating the relative positions of the HLA-DRA promoter, the RFX binding site, the luciferase gene, the HgaI and AvaI restriction enzyme sites used in the methylation sensitive Real-Time PCR experiments of panels F-I, and of primers a-f used in the Real-Time PCR experiments.

FIG. 4E shows the HLA-DRA promoter sequence of pDRA indicating SssI CG methylation sites (in larger font) and the binding sites of RFX, Oct-1 and YY1.

The X2- and Y-elements are also required for HLA-DRA promoter activity.

FIGS. 4f through 4I are graphs of experiment results showing methylation sensitive Real-Time PCR.

FIGS. 5A and 5B are graphs of experiment results showing the Oct-1 and YY1 repression mechanisms do not function with methylated pDRA. 5B. Repeat of FIG. 5A, except with methylated pDRA (Me-pDRA), methylated pDRAΔOct (Me-pDRAΔOct), or methylated pDRAΔYY1 (Me-pDRAΔYY1).

FIGS. 5C and 5D are graphs of experiment results showing two examples of a repeat of FIG. 5B except the Me-pDRA and Me-pDRA mutated constructs were transfected with the RFX expression vectors.

FIGS. 6A and 6B are two models for how RFX could mediate enhanceosome formation on methylated HLA-DRA promoter DNA. MBD, methyl DNA binding domain protein; HDAC, histone deacetylase; Nuc, nucleosome.

FIG. 7 is a graph of pDRA HgaI methylation sensitive PCR.

FIG. 8 represents repressosome and enhanceosome states and transitions for methylated and nonmethylated HLA-DRA promoter DNA.

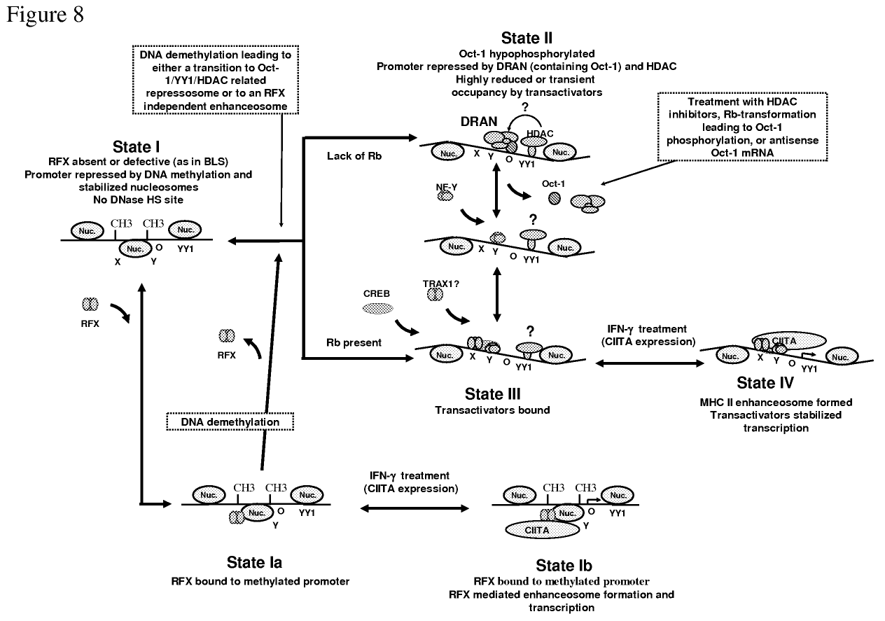

### DETAILED DESCRIPTION OF THE PREFERRED EMBODIMENT

In the following detailed description of the preferred embodiments, reference is made to the accompanying drawings, which form a part hereof, and within which are shown by way of illustration specific embodiments by which the invention may be practiced. It is to be understood that other embodiments may be utilized and structural changes may be made without departing from the scope of the invention.

As used herein; a “test agent” refers to an agent that is to be screened in one or more of the assays described herein. The agent can be virtually any compound and can exist as a single isolated compound or can be a member of a chemical library.

As used herein; a “reporter gene” refers to any gene or DNA that expresses a product that is detectable by spectroscopic, photochemical, biochemical, enzymatic, immunochemical, electrical, optical or chemical means. The preferred reporter gene to which a promoter element is ligated is luciferase. Other reporter genes for use for this purpose include, for example, .beta.-galactosidase gene (.beta.gal) and chloramphenicol acetyltransferase gene (CAT) Assays for expression produced in conjunction with each of these reporter gene elements are well-known to those skilled in the art. Assay in connection with the luciferase gene are described below.

Expression constructs are prepared by ligating transcriptional promoter elements to the reporter genes by methods well-known in the art, e.g., by utilizing restriction enzymes to cut the reporter gene in appropriate portion to provide binding sites for the transcriptional promoter elements, incubating the restriction enzyme treated reporter gene with the transcriptional promoter elements and screening for the recombinants.

The term “gene of interest” or “target gene” refers to a nucleic acid which can be of any origin and isolated from a genomic DNA, a cDNA, or any DNA encoding a RNA, such as a genomic RNA, a mRNA, an anti-sense RNA, a ribosomal RNA, a ribozyme or a transfer RNA. The gene of interest can also be an oligonucleotide (i.e., a nucleic acid having a short size of less than 100 bp). It can be engineered from genomic DNA to remove all or part of one or more intronic sequences (i.e., minigene).

In a one embodiment, the gene of interest in use in the present invention, encodes a target gene product, such as a protein, of therapeutic interest. A gene product of therapeutic interest, or target gene protein, is one which has a therapeutic or protective activity when administered appropriately to a patient, especially a patient suffering from a disease or illness condition or who should be protected against this disease or condition. Such a therapeutic or protective activity can be correlated to a beneficial effect on the course of a symptom of said disease or said condition. It is within the reach of the man skilled in the art to select a gene encoding an appropriate gene product of therapeutic interest, depending on the disease or condition to be treated. In a general manner, his choice may be based on the results previously obtained, so that he can reasonably expect, without undue experimentation, i.e., other than practicing the invention as claimed, to obtain such therapeutic properties.

Herein, the inventors show that the sequence specific binding protein, RFX, mediates the transcriptional activation of a methylated major histocompatibility (MHC) gene promoter that was repressed by the methylation. This indicates that DNA demethylation is not a necessary step in promoter activation and shows that methylated DNA can be transcribed. Furthermore, the RFX-related results show that a sequence specific DNA binding protein can mediate a transition between a repressed and active promoter.

The methyl-DNA binding protein, RFX, was first identified as a protein defective in Bare Lymphocyte Syndrome, a rare immunodeficiency disease that is due to the lack of transcription of the MHC class II genes, which encode the class II antigen presenting molecules. RFX has two apparent functions in facilitating MHC class II promoter activation. RFX participates in the formation of an MHC class II enhanceosome, by binding specifically to the MHC class II promoter and interacting with other MHC class II promoter binding proteins as well as with the MHC class II specific coactivator (CIITA).

RFX also mediates the establishment of an MHC class II DNase I hypersensitive site, thus facilitating a transition from an inactive, condensed form of chromatin to a form of promoter chromatin that permits enhanceosome assembly and transcriptional activation. RFX5, one of the three subunits of RFX, is a member of a family of methyl-DNA binding proteins, some of which also have a connection with the regulation of transcription of DNA that is in a condensed form.

For example, drosophila RFX functions in sperm, and RFX2 and RFX4 function in the testis.

### EXAMPLE I

### DNA Methylation Represses Transcriptional Activity of the HLA-DRA Promoter

Repression of Endogenous HLA-DRA Promoter

B-cells specifically lacking RFX, but possessing the other required transactivators, do not have a transcriptionally active HLA-DRA gene (the human MHC class II gene that represents the prototype for MHC class II gene regulation).

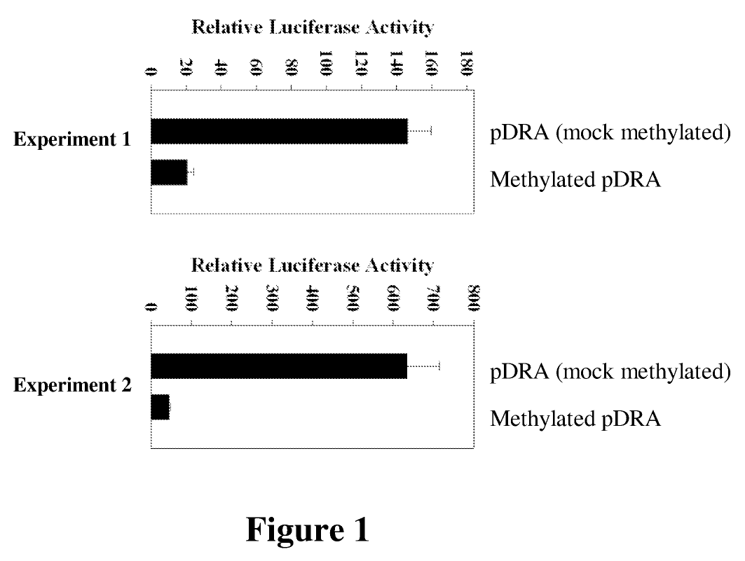

RFX-defective B-cells have a condensed, inaccessible HLA-DRA promoter chromatin conformation. The inventors treated two RFX-negative cell lines with Azacytidine (AzaC), which inhibits DNA methyltransferase activity, and assayed the cells for HLA-DRA mRNA by RT-PCR (FIG. 1A) to determine whether DNA methylation plays a role in preventing transcription of HLA-DRA in RFX-negative cells.

Suspension B-cell lines were seeded at 10⁵ cells per ml in 20 ml volume of media. Cells were treated with 5 uM Azacytidine for 72 hours. Total RNA was prepared as described, and 5 ug of RNA were used in a standard reverse transcriptase, polymerase chain reaction (RT-PCR) for HLA-DRA mRNA and for γ-actin mRNA. PCR was performed for 40 cycles and the PCR products detected by 2.2% agarose gel electrophoresis.

AzaC treatment led to an increase in HLA-DRA mRNA production compared to untreated cells. This increase did not lead to mRNA production levels seen in RFX-positive B-cells, as expected, because the AzaC does not rescue the enhanceosome function of RFX. However, these results indicate that RFX-negative cells have a methylated HLA-DRA gene and that the methylation plays a role in preventing HLA-DRA transcription.

Repression of Transfected HLA-DRA Promoter

The inventors methylated a pDRA (luciferase) construct with SssI methylase, which methylates all plasmid CpG dinucleotides, to determine whether an HLA-DRA promoter-reporter construct could be repressed by methylation. Methylated pDRA was transfected into 5637 cells, which require IFN-γ treatment for the activation of the pDRA promoter.

The HLA-DRA expression construct (promoter-luciferase), pDRA derived from pGL3, was methylated using SssI methylase (20 ug pDRA, 5 mM S-adenosyl methionine (SAM), 8 units of SssI, 10× New England BioLabs buffer #2, H₂O to a total volume of 50 ul). Mock methylated pDRA was prepared in the same way but without SssI methylase in the reaction. Methylated pDRA, or mock methylated DNA, was extracted with phenol:chloroform, ethanol precipitated, resuspended in water, and quantified by absorbance at A₂₆₀ and by agarose gel electorphoresis. Fifty nanograms of methylated pDRA were added to each of six wells containing 5×10⁴ 5637 bladder carcinoma cells. DNA transfection was performed using the Transit Reagent according to the vendor's instructions. IFN-γ was added to 400 units/ml following the transfection and luciferase assays were performed 24 hours following the transfection. Each bar graph, FIG. 1, represents the average and standard deviations for six transfections. The SssI methylation significantly reduced the IFN-γ activation of the pDRA (FIG. 1).

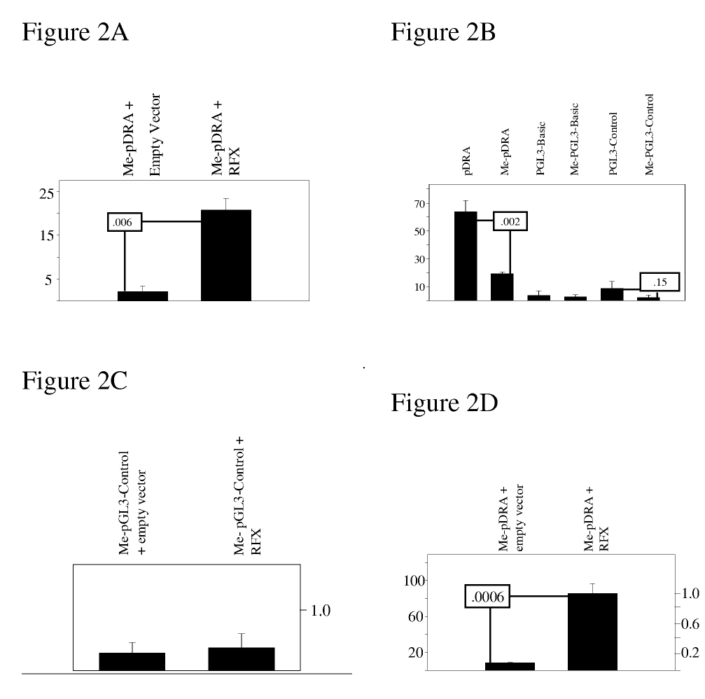

Methylated pDRA is insensitive to AvaI digestion. pDRA has two AvaI sites: one site in the HLA-DRA promoter region cloned upstream of the luciferase coding region and one site in the luciferase coding region. (See pDRA map, FIG. 2D.)

### EXAMPLE II

### RFX Facilitates Activation of a Methylated HLA-DRA Promoter Reporter Construct

Methylated pDRA was co-transfected into 5637 cells with equal amounts of either (a) empty vector or (b) the RFXAP, RFXB, and RFX5 expression vectors, representing the three subunits of RFX. Cells were treated with IFN-γ for 24 hours following transfection. The methylated pDRA co-transfected with RFX showed a ten-fold increase (FIG. 2A) in luciferase activity compared with the controls samples lacking the exogenous RFX. Fifty (50) ng of methylated pDRA (Me-PDRA) was co-transfected with 50 ng each of CMV-based expression vectors for RFX5, RFXAP and RFXB (also termed RFXANK or Tv1-1), representing the RFX trimer, or with 150 ng of empty expression vector, into 5×10⁴ 5637 cells. Following the transfection, cells were treated with IFN-γ. Luciferase averages and p-values were obtained by using six wells for each transfection. Quantification of the luciferase assay is indicated to the left of the bar graphs and the p-value is indicated in boxes.

To be certain that the increase in transcriptional activity was caused by RFX binding to the HLA-DRA promoter sequences of pDRA, we assessed the effect of RFX on pGL3 promoter reporter constructs lacking any known RFX sites or lacking any apparent sites for the other sequence specific DNA binding proteins in the MHC class II enhanceosome. We methylated the pGL3-Basic and pGL3-Control luciferase constructs and transfected them into cells. pGL3-Basic, lacking any known promoter elements, had very little activity whether methylated or unmethylated. Methylation of the pGL3-Control luciferase construct, which contains the SV40 enhancer, reduced its promoter activity. The methylated or mock methylated promoter luciferase constructs were transfected into cells as indicated above. The pGL3-Basic (with no promoter activity) was used to construct pDRA. The pGL3-Control contains the SV40 promoter and enhancer. P-values are indicated in boxes (FIG. 2B).

RFX was co-transfected with methylated pDRA (Me-pDRA) as a positive control for the experiment (FIG. 2C). Relative luciferase activity is indicated to the right of the bar graphs, with the results from pDRA co-transfected with RFX arbitrarily set at 1.0, for comparison with panel C. Actual luciferase quantification is to the left of the bar graphs and the p-value is indicated in the box. The methylated pGL3-Control luciferase construct was then co-transfected into cells with the RFXAP, RFXB, and RFX5 expression vectors or with empty vector.

Methylated pDRA co-transfected with the RFX expression vectors represented a positive control for the RFX effect (FIG. 2D).

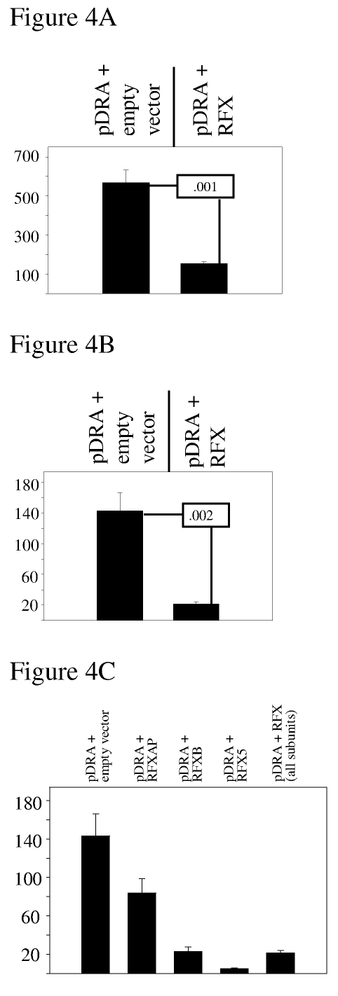

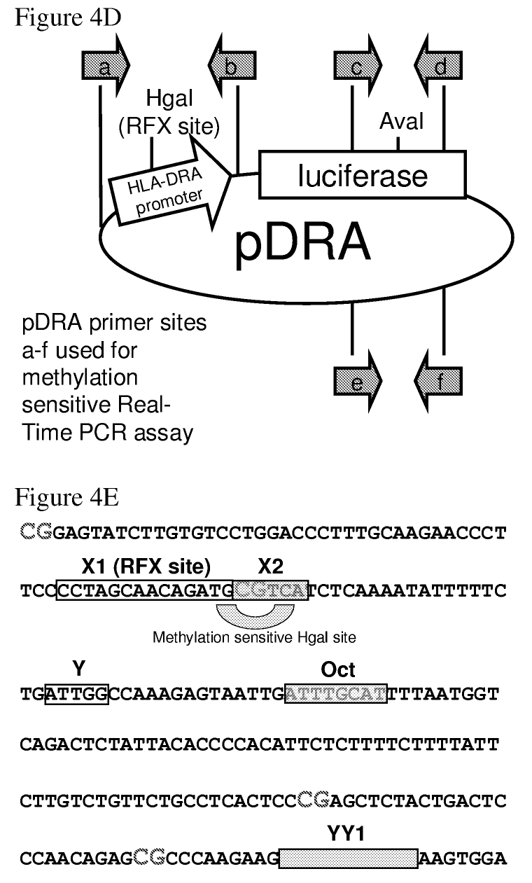

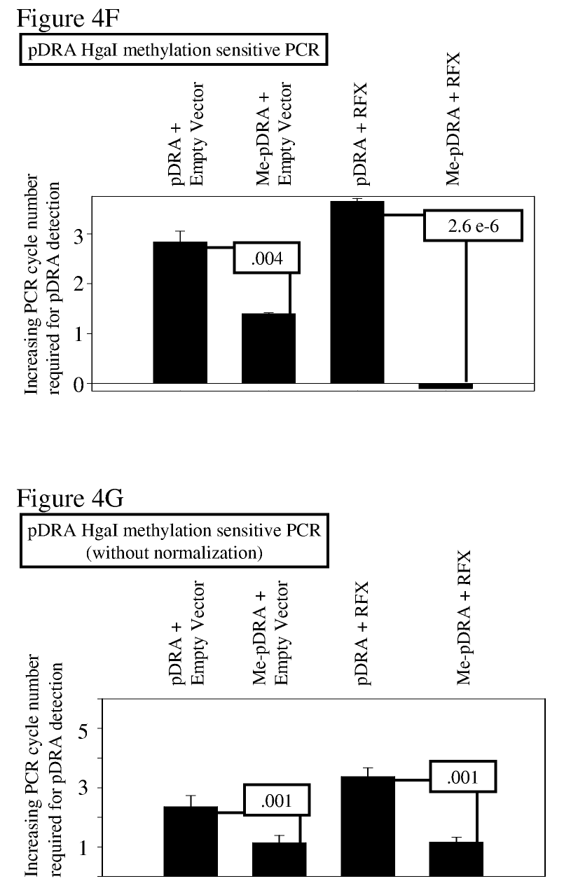

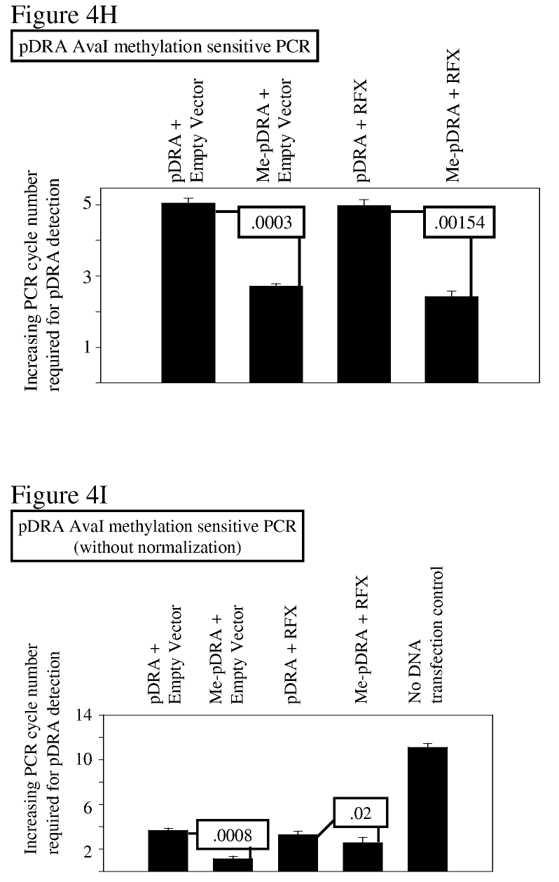

Methylated pGL3-Control (Me-pGL3 Control) co-transfected with the RFX expression vectors, or with an equivalent amount of empty vector. Relative luciferase activity is indicated to the right (FIG. 2C). Thus, results indicated essentially no increase in activity due to RFX expression. No increase in transcriptional activity was observed for the pGL3-Control luciferase construct (FIG. 2C); consistent with the conclusion that RFX activates methylated pDRA by binding to its cognate site in the DRA promoter (FIG. 4E).

### EXAMPLE III

### RFX is Required for the CIITA Activation of Methylated pDRA

In general, the sequence specific DNA binding proteins can occupy the HLA-DRA promoter without the advent of HLA-DRA transcription. Transcriptional activation commences when the formation of the enhanceosome, including RFX, is completed by availability and the enhanceosome binding of CIITA, which in turn leads to the recruitment of histone deacetylase activity. CIITA synthesis is induced by IFN-γ, as a result of the IFN-γ induction of activated STAT1 and IRF-1 and the binding of these two transactivators to the CIITA promoter.

To be certain that RFX facilitated the IFN-γ activation of the methylated pDRA promoter by cooperating with CIITA, rather than by any unappreciated indirect effect, the inventors co-transfected the methylated pDRA with a CIITA expression vector and with either the RFX expression vectors or with control empty vector.

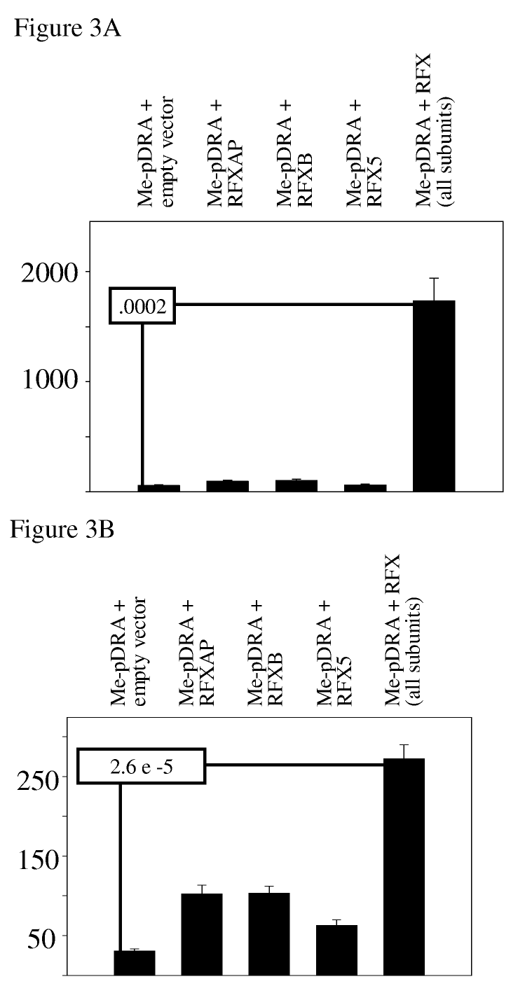

Referring now to FIG. 3, Panel A. (i) Methylated pDRA (Me-pDRA), (ii) the RFX expression vectors or empty vector as indicated, and (iii) a CIITA expression vector were co-transfected as described in FIGS. 1 and 2, except 50 ng of CIITA was used for each of six wells and the cells were not treated with IFN-γ. P-value is indicated in the box. Panel B. shows a separate, duplicate experiment, same as panel A. Greater absolute luciferase activity is observed with CIITA activation of pDRA vs. IFN-γ activation in FIGS. 1 and 2. RFX expression strongly enhanced the CIITA activation of the methylated pDRA.

### EXAMPLE IV

### RFX Does Not Facilitate the Activation of Nonmethylated pDRA and Does Not Facilitate Demethylation of pDRA

While RFX is capable of low affinity binding to nonmethylated HLA-DRA promoter DNA and of participating in enhanceosome formation in vitro in the absence of DNA methylation, it is not known whether RFX is important in the activation of nonmethylated DNA. The inventors co-transfected either the RFX expression vectors or empty vector with nonmethylated pDRA (FIGS. 4A, B, C).

Referring to FIG. 4A, cells were co-transfected with mock methylated pDRA and either empty vector or the three RFX expression vectors. P-value indicated in the box. FIG. 4B is a repeat of the experiment in FIG. 4A. FIG. 4C is a repeat of the experiment in FIG. 4A, except cells were co-tranfected with CIITA instead of treated with IFN-γ and with individual RFX expression vectors as indicated.

These experiments indicated that the same amounts of the expression vectors used to demonstrate that RFX facilitates CIITA activation of methylated DNA lead to decreased HLA-DRA promoter activity for nonmethylated pDRA. This result indicates that another protein, rather than RFX, mediates the X1 HLA-DRA promoter element (RFX promoter binding site; FIG. 4E) function for nonmethylated DNA, possibly TRAX1, previously reported to bind the X1 element and activate nonmethylated HLA-DRA promoter DNA in vitro

To rule out the possibility that RFX facilitated demethylation of the pDRA, the inventors recovered total DNA from cells co-transfected with either the RFX expression vectors or the empty vector in combination with either methylated pDRA or nonmethylated pDRA. The recovered DNA was digested with either HgaI, the site for which overlaps the CG dinucleotide of the RFX binding site (X1 element) in the HLA-DRA promoter (FIG. 4E), or with AvaI. Neither HgaI nor AvaI cleave DNA when the C at the cleavage site is methylated. The digested DNA was then assayed by Real-Time PCR using pDRA specific primers on either side of the pDRA promoter HgaI site or luciferase coding sequence primers on either side of an AvaI site located in the luciferase coding region (FIG. 4D).

Twenty four hours following transfection of the indicated plasmids, total cellular DNA was isolated. Briefly, cells from a 100 mm plate were washed with PBS, lysed in SDS buffer, and the DNA was sheared by passage of the cell lysate through a 20 gauge syringe exactly ten times. The cell lysate was incubated with proteinase K and RNase, and DNA was extracted using phenol/chloroform. The DNA was ethanol precipitated, resuspended in water and quantified. Equal amounts of DNA (about 1% of the DNA recovered from a 100 mm plate) were digested with the indicated methylation sensitive restriction enzymes and were amplified by Real-Time PCR.

Primers e and f were used to determine the relative amounts of transfected plasmid present for each of the indicated transfections, as these primers amplify a segment of pDRA which does not include a site for either HgaI or AvaI. The results for FIGS. 4F and 4H were obtained by normalizing the results for each transfection using the results obtained with primers e and f. Thus, the relatively low number of cycles obtained for primers e and f, covering a region of pDRA unaffected by the restriction enzymes (FIG. 4D) and thus representing all of the available amplifiable DNA, for each transfection, were subtracted from the number of cycles obtained for primers a and b in FIG. 4F and from the number of cycles obtained for primers c and d in FIG. 4H. In sum, for FIGS. 4F and 4H, the y-axis numbers represent the additional loss of plasmid due to cutting by the methylation sensitive enzymes.

The results for FIGS. 4G and 4I were not normalized and, in the case of FIG. 4I, include a “no-transfection control”. The y-axis numbers for the FIG. 4H bar graph represent relative cycle numbers where the methylated pDRA co-transfected with empty vector was set to “1” for convenience in comparing the results for each set of transfected DNAs. The actual cycle numbers for all Real-Time PCR results in FIGS. 4F-4I ranged from 21-26 with the exception of the “no-transfection” control in FIG. 4H which averaged about 33 cycles. P-values are shown in boxes. The maximum cycle number difference between methylated and nonmethylated DNA, based on the digestion and amplification of methylated or nonmethylated pDRA prepared in vitro, rather than extracted from cells following a transfection, is four cycles (data not shown). Thus, the increase in the HgaI sensitivity of pDRA vs. methylated pDRA, when co-transfected with RFX, indicated in FIG. 4F as about four cycles, is close to or at the theoretical maximum. This in turn indicates: (i) that little or no demethylation at this site occurs during the period of DNA transfection and RFX expression; (ii) that about half of the DNA is demethylated at the HgaI site in the absence of RFX; and (iii) that about half of the DNA is demethylated at the luciferase AvaI site in the presence or absence of RFX. In sum, these data indicate that RFX expression does not facilitate pDRA demethylation.

These methylation sensitive PCR results indicated that nonmethylated DNA was significantly more sensitive than the methylated DNA to both of the restriction enzymes. Furthermore, the results indicated that RFX expression did not lead to an increase in DNA demethylation compared with the empty vector (FIGS. 4F-4I), and thus the activation of the methylated pDRA by RFX (FIGS. 2, 3) cannot be explained by RFX mediated DNA demethylation. Interestingly, the results of the methylation sensitive PCR also indicated that RFX expression stabilizes the methylation of the C in the CG dinucleotide that is immediately adjacent to the apparent RFX binding site (FIGS. 4D, 4E) relative to the methylation of the C in the AvaI site distal to the RFX binding site (FIG. 4F-4I).

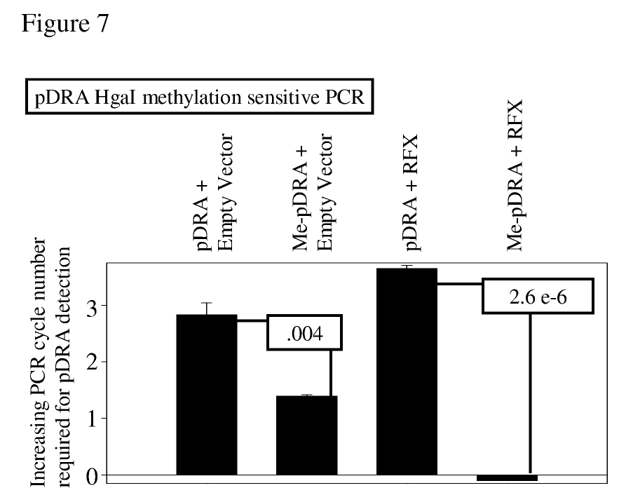

Because RFX does not facilitate the CIITA activation of nonmethylated DNA or an increase pDRA demethylation, the RFX activation of methylated pDRA must be due to RFX binding to methylated pDRA and to the RFX-mediated recruitment of CIITA to the methylated pDRA (FIG. 7). CIITA in turn facilitates the recruitment of HAT activity to the methylated DNA, the repression of which is otherwise maintained by deacetylated histones. These results represent the first description of a molecular mechanism indicating how a specific, methylated promoter can transition from a repressed to an activated state. The process of RFX mediated enhanceosome formation on the methylated HLA-DRA promoter suggests that the transition from methylated repressed DNA to methylated, activated DNA does not require a nonspecific transition over long regions of heterochromatin.

### EXAMPLE V

### The Oct-1/YY-1 Repression Mechanism Does Not Function to Repress Either Inactive or Transcriptionally Competent Methylated pDRA

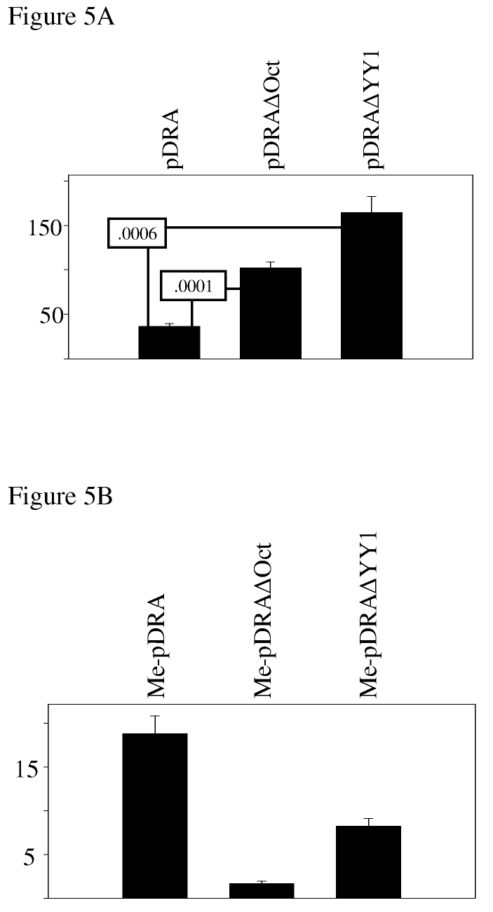

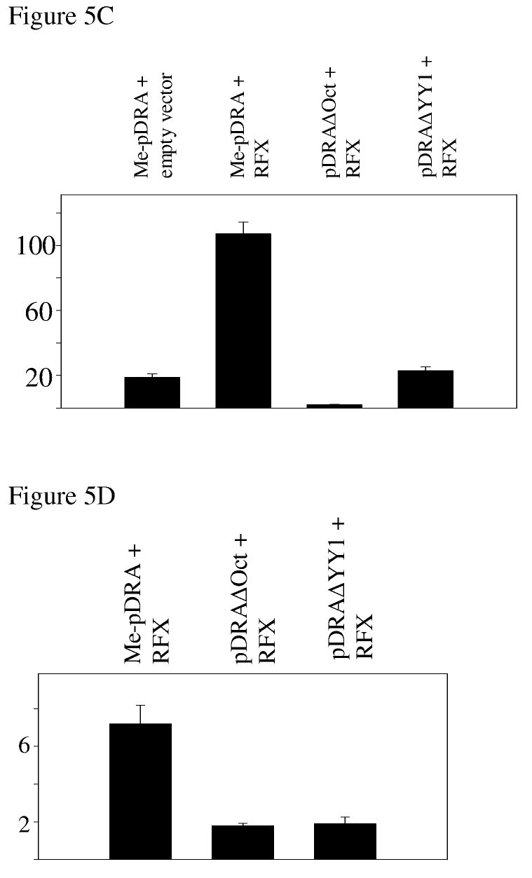

Both Oct-1 and YY-1 repress the IFN-γ activation of pDRA. To determine whether this mechanism(s) functions to represses methylated pDRA, the inventors transfected methylated pDRA or the previously described, methylated pDRAΔOct or methylated pDRAΔYY1 into IFN-γ treated 5637 cells. The latter two pDRA luciferase constructs lack the Oct-1 and YY1 binding sites respectively. The inventors also transfected nonmethylated pDRA or pDRAΔOct or pDRAΔYY1 into IFN-γ treated 5637 cells, a transfection series that serves as a positive control for the detection of Oct-1 or YY1 mediated repression. Mock methylated pDRA or mock methylated pDRA mutants lacking the Oct-1 binding site or the YY1 binding site were transfected into cells and assayed for luciferase activity. Results indicate that the nonmethylated DNA is repressed by Oct-1 and YY1 (FIG. 5A).

The repression is revealed by an increase in luciferase activity when comparing the activity of either pDRAΔOct or pDRAΔYY1 to pDRA. However, the lack of these sites does not lead to increased activity when pDRA is methylated, with or without exogenous RFX expression (FIGS. 5B-5C).

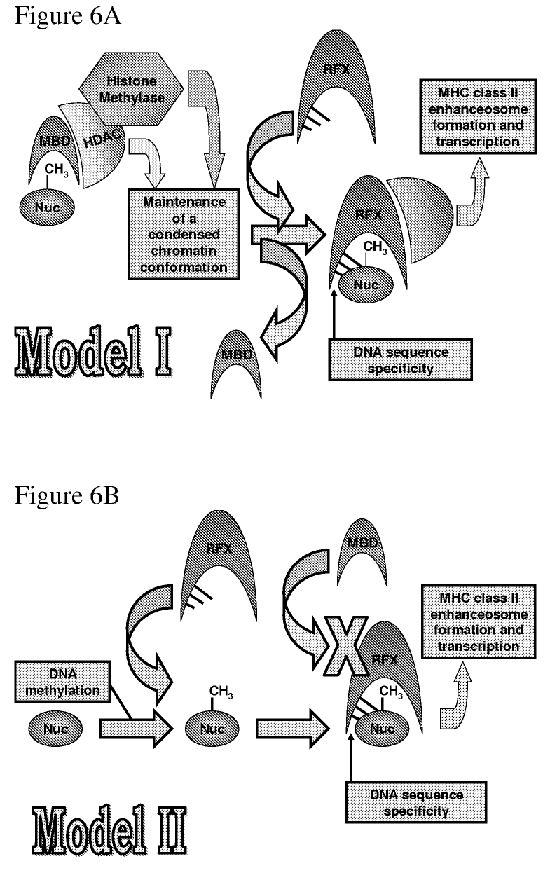

The data shows for the first time that a sequence specific DNA binding protein can support enhanceosome formation on, and transcriptional activation of methylated promoter DNA; that localized, gene-specific transcription of methylated DNA is possible; that RFX facilitates activation of a methylated HLA-DRA promoter but not a demethylated HLA-DRA promoter; and that function of the Oct-1 and YY1 repression mechanisms require demethylated DNA, despite the fact that methylated DNA can be transcriptionally active. These data and conclusions further support the idea that a transition from repression that is mediated by methylation of DNA to transcriptional activation need not require demethylation. Thus, RFX transactivator may either displace or prevent the binding of MBD proteins that form a repressosome complex (FIG. 6), leading to promoter activation.

The described mechanism of transitioning from a repressed methylated promoter to an activated methylated promoter raises the question of whether a transition from methylated DNA to demethylated DNA is instigated by mRNA transcription? If so, the role of transcription in facilitating DNA demethylation would be reminiscent of other DNA modifications that are stimulated by transcription, such as immunoglobulin class switching and DNA repair.

The HLA-DRA promoter binding specificity for RFX requires all three subunits. Multiple RFX5 family members (RFX1-5) have been well-studied and have related “RFX-type” DNA binding domains. Because all three subunits of RFX are required for HLA-DRA promoter specificity, it is not likely that significant sequence specificity resides in the RFX-type DNA binding domain common to these RFX5 family members. The other two RFX subunits, RFXAP and RFB/RFXANK/Tv1-1, have no known functional or structural homologs. However, because these subunits are required for HLA-DRA promoter binding specificity of the RFX trimer, it is likely that other, currently unknown proteins substitute for RFXAP and RFXANK in establishing sequence specificity for enhanceosomes that form on other methylated promoters.

This invention has implications for transitions between different repressosomes, and in particular indicate that demethylation of DNA is likely to represent a mechanism of transitioning between repressosomes. For example, demethylation of the HLA-DRA promoter represents a transition from the methyl DNA binding protein-related repressosome that regulates methylated HLA-DRA DNA (FIG. 1) to the DRAN (Oct-1 related) or YY1-HDAC repressosomes that regulate demethylated HLA-DRA DNA (FIG. 7).

It will be seen that the objects set forth above, and those made apparent from the foregoing description, are efficiently attained and since certain changes may be made in the above construction without departing from the scope of the invention, it is intended that all matters contained in the foregoing description or shown in the accompanying drawings shall be interpreted as illustrative and not in a limiting sense.

It is also to be understood that the following claims are intended to cover all of the generic and specific features of the invention herein described, and all statements of the scope of the invention which, as a matter of language, might be said to fall there between. Now that the invention has been described,

What is claimed is:

1. A method of identifying a transcriptional activation mediator of a methylated gene of interest, comprising the steps of:
providing a cell comprising a methylated construct further comprising a reporter-gene operably linked to a promoter of the gene of interest;
administering a test agent to the cell; and
comparing the expression of the reporter gene to a control to determine the test agent's ability to mediate transcriptional activation of the methylated gene.

2. The method of claim 1 wherein the reporter gene is selected from the group consisting of luciferase, β-galactosidase, chloramphenicol acetyl transferase (CAT), green fluorescent protein, and red fluorescent protein.

3. The method of claim 1 wherein the test agent is a DNA binding protein.

4. The method of claim 3 wherein the DNA binding protein is methylated.

5. The method of claim 1 wherein the expression construct is pDNA.

6. The method of claim 1 wherein the expression construct is methylated with SssI Methylase.

7. A method of identifying a gene of interest capable of methylation-dependent activation, comprising the steps of;
providing a cell comprising the gene of interest;
establishing the gene of interest is transcriptionally inactive responsive to DNA methylization associated therewith;
administering a methylated expression construct to the cell, comprising a promoter of the gene of interest operably linked to a reporter gene;
determining the expression of the gene of the interest, compared to a control, wherein a comparative increase in the expression of the gene of interest in the cell indicates methylation-dependent activation thereof.

8. The method of claim 7 wherein the step of establishing the gene of interest is transcriptionally inactive responsive to DNA methylization associated with a promoter thereof further comprises the steps of;
providing a cell comprising the gene of interest;
contacting the cell with a DNA methyltransferase inhibitor; and
assaying the cell for mRNA associated with the gene of interest wherein an increase in the production of mRNA associated with the gene of interest indicates the repressed gene of interest is transcriptionally inactive responsive to DNA methylization associated with a promoter thereof.

9. The method of claim 8 wherein the DNA methyltransferase inhibitor is Azacytidine.

10. The method of claim 7 wherein the step of establishing the gene of interest is transcriptionally inactive responsive to DNA methylization associated with a promoter thereof further comprises the steps of;
providing a methylated expression construct, comprising a promoter of the gene of interest operably linked to a reporter gene;
providing a test-cell requiring an enhancing-agent for activation of the promoter;
administering the methylated expression construct to the test cell;
administering an enhancing agent associated with activation of the promoter to the test-cell; and
assaying the cell for mRNA associated with the gene of interest wherein a decrease in the production of mRNA associated with the gene of interest indicates the gene of interest is transcriptionally inactive responsive to DNA methylization associated therewith.

11. Method of claim 10 wherein the enhancing agent is IFN-γ.

12. A method of screening for an agent as a candidate for treatment of inflammation associated with the expression of a methylated gene of interest, comprising the steps of:
providing a cell comprising a methylated construct further comprising a reporter-gene operably linked to a promoter of the gene of interest;
transfecting the cell with a DNA binding protein specific to the gene of interest;
contacting the cell with a test agent; and
measuring the expression of the reporter gene compared to a control as an indicator of the test agent's ability to modulate expression of the gene of interest.

13. The method of claim 11 further comprising the step of contacting the cell with an enhancing agent associated with the promoter after transfecting the cell with a DNA binding protein specific to the gene of interest.

14. The method of claim 12 wherein the enhancing agent is IFN-γ.

15. The method of claim 11 wherein the gene of interest is HLA-DRA.

16. The method of claim 11 wherein the reporter gene is luciferase.

17. The method of claim 11 wherein the DNA binding protein is methylated.

18. The method of claim 16 wherein the methylated DNA binding protein is RFX.

19. The method of claim 11 wherein the cell lacks RFX.

20. The method of claim 11 wherein the test agent is a putative inhibitor of HLA-DRA gene expression.
